(19) United States
(12) Patent Application Publication
(10) Pub. No.: US 2006/0147434 A1
(43) Pub. Date: Jul. 6, 2006

(54) Different dendritic cell subsets

(76) Inventors: Kazumasa Ogasawara, Otsu-shi (JP); Kaiichi Kajino, Kusatsu-shi (JP); Ichiro Nakamura, Otsu-shi (JP); Keiichi Kontani, Otsu-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/542,104

(22) PCT Filed: Jan. 16, 2004
(86) PCT No.: PCT/JP04/00332

§ 371 Date: Mar. 9, 2006

(30) Foreign Application Priority Data
Jan. 16, 2003 (JP) 2003-008807

(51) Int. Cl. A61K 48/00; C12N 5/08

(52) U.S. Cl. 424/93.21; 435/372

(57) ABSTRACT

It is intended to provide expired dendritic cells having characteristics of the following (E1) to (E3): (E1) not shifting into a mature type due to an action of a natural immune stimulant or a permanent immune potentiator;(E2) having the same shape as immature DC; and (E3) expressing IL-10. It is intended to provide Permanently activated dendritic cells having the following characteristics:(M2-1) having projecting dendrites and forming aggregation clusters;(M2-2) being capable of activating unreacted cytotoxic T cells (CTL);(M2-3) having stable properties under the action of anti-CD40 monoclonal antibody; and (M2-4) showing a high expression level of at least one member selected from the group consisting of CD80, CD83 and CD86.

### TECHNICAL FIELD

The present invention relates to expired dendritic cells, a method for transient or permanent maturation of dendritic cells, an anti-cancer agent and an immunosuppressive drug using these dendritic cells, further a method for treating cancer and a method for transplanting an organ or a tissue.

Herein, the dendritic cell is sometimes abbreviated as “DC”.

### BACKGROUND ART

A dendritic cell (DC) is an important mediator between natural immunity and adaptive immunity. Focusing on inflammatory, endotoxin or inflammatory cytokine associated with the natural immunity induces differentiation of immature DC into mature DC. The latter efficiently stimulates helper T cells and cytotoxic T cells which are major effectors in the adaptive immunity (Banchereau, J. & Steinman, R. M. Nature 392, 245-252 (1998); Mellman, I. & Steinman, R. M. Cell 106, 255-258 (2001)). Stimulation by CD40 ligand (CD154) present on activated T cells to the immature DC via CD40 give a signal for DC maturation. However, relative importance of various factors involved in the DC maturation has remained unclear yet. Only pipetting or replating the cells in accordance with an original induction protocol described for the preparation of the immature DC from bone marrow caused the maturation (Inaba, K. et al J. Exp. Med. 191, 927-936 (2000); Gallucci, S., Lolkema, M. & Matzinger, P. Nat. Med., 5, 1249-1255 (1999)).

References 1 to 22 are further included as literatures known publicly associated with the present invention.

However, these publicly known references have not shown an overall picture of the process for the DC maturation.

### DISCLOSURE OF THE INVENTION

It is an object of the present invention to provide new findings for maturation process of DC.

Specifically, it is the object of the invention to provide expired dendritic cells having an immunosuppressive function, mature DC (M2DC: mature 2 dendritic cells) having a permanently activated immune function and methods for the preparation thereof, and a method for preparing transiently activated mature DC (M1DC: mature 1 dendritic cells) having an immunostimulatory function.

It is another object of the present invention to provide an immunosuppressive drug, an anti-cancer agent, a method for treating cancer and a method for transplanting an organ or a tissue where an immunologic rejection is inhibited.

The present inventors have evaluated the maturation of immature DC (dendritic cells) using natural immune stimulants such as endotoxin (LPS), anti-CD40 monoclonal antibody (mAb) (alternative of CD154), TNFα (example of proinflammatory cytokine) and picibanil (OK432) without pipetting and replating as the above.

The present inventors will describe expired DC induced by a natural immune stimulant. This expired DC is similar to the immature DC in many points except for expression of MHC class I at a high level, production of IL-10 and non-reactivity to the stimulation which induces the maturation in another way. such expired DC can not stimulate unreacted cytotoxic T cells (CTL). Rather, the expired DC induce anergy in a CTL clone and have an immunosuppressive action. The stimulation via CD40 inhibited LPS-induced shift into an expired phenotype, and consequently a mature phenotype which was different from a phenotype observed in an early stage after the LPS stimulation was acquired, i.e., the shift into M2DC was brought.

The present inventors have also observed that the immature DC migrate into regional lymph nodes after being stimulated with the natural immune stimulant and interact with activated helper T cells there, thereby the mature phenotype is maintained. Based on these data, the present inventors will propose a novel concept for the DC maturation involved in immune regulation. The present inventors have demonstrated the DC maturation process in detail for the first time.

That is, the present invention relates to the followings.

[1] An expired dendritic cell having the following characteristics (E1) to (E3):
(E1) not shifting into a mature type due to an action of a natural immune stimulant or a permanent immune potentiator;
(E2) having the same shape as immature DC; and
(E3) expressing IL-10.

[2] The expired dendritic cell according to [1] wherein the dendritic cell is a human dendritic cell.

[3] The human expired dendritic cell according to [2] having the following characteristics:
(E1′) not shifting into a mature type due to an action of LPS and anti-CD40 monoclonal antibody;
(E2) having the same shape as immature DC; and
(E3) expressing IL-10.

[4] The human expired dendritic cell according to [3] further having the following characteristics:
(E4) having an expression level of CD80 nearly equivalent to that on the immature DC; and/or
(E5) having an expression level of CD83 nearly equivalent to that on the immature DC.

[5] The human expired dendritic cell according to [4] further having at least one of the following characteristics:
(E6) having a phagocytic activity for microbeads nearly equivalent to that of the immature dendritic cells;
(E7) expressing MHC class I at a high level;
(E8) not activating unreacted T cells in the presence of an antigenic peptide; and
(E9) expressing TLR4/MD2 at a lower level than the immature DC.

[6] Permanently activated dendritic cells having the following characteristics:
(M2-1) having projecting dendrites and forming aggregation clusters;
(M2-2) being capable of activating unreacted cytotoxic T cells (CTL);
(M2-3) having stable properties under the action of anti-CD40 monoclonal antibody; and
(M2-4) showing a high expression level of at least one member selected from the group consisting of CD80, CD83 and CD86.

[7] The permanently activated dendritic cells according to [6] wherein the dendritic cell is a cell derived from human, having the following characteristics:
(M2-1) having projecting dendrites and forming aggregation clusters;
(M2-2) being capable of activating unreacted cytotoxic T cells (CTL);
(M2-3) having stable properties under the action of anti-CD40 monoclonal antibody; and
(M2-4′) expressing CD80 and CD83 at a high level.

[8] The permanently activated dendritic cells according to [7] further having at least one of the following characteristics:
(M2-5) expressing FcγR at a low level (FcγR^{low});
(M2-6) expressing MHC-I at a high level (MHC-I^{high});
(M2-7) expressing MHC-II at a high level (MHC-II^{high}); and
(M2-8) expressing IL-12 p40 at a high level.

[9] A method for preparing expired dendritic cells (expired DC) comprising a step of activating immature dendritic cells with a natural immune stimulant to induce transiently activated mature dendritic cells (M1DC), and a step of culturing the M1DC in the absence of a permanent immune potentiator.

[10] A method for preparing permanently activated mature dendritic cells (M2DC) comprising a step of treating immature dendritic cells with a permanent immune potentiator.

[11] A method for preparing permanently activated mature dendritic cells (M2DC) comprising a step of activating immature dendritic cells with a natural immune stimulant to induce transiently activated mature dendritic cells (M1DC), and a step of culturing the M1DC in the presence of a permanent immune potentiator.

[12] A method for preparing transiently activated mature dendritic cells (M1DC) characterized by treating immature dendritic cells with a natural immune stimulant.

[13] An anti-cancer agent wherein the human permanently activated dendritic cell (M2DC) according to [7] or [8] or the human M2DC prepared by the method according to [10] or [11] is an active ingredient.

[14] An anti-pathogen agent wherein the human permanently activated dendritic cell (M2DC) according to [7] or [8] or the human M2DC prepared by the method according to [10] or [11] is an active ingredient.

[15] An immunosuppressive drug wherein the expired dendritic cell according to [1] to [5] or the expired dendritic cell obtained by the method according to [9] is an active ingredient.

[16] A method for treating cancer characterized in that the human permanently activated dendritic cell (M2DC) according to [7] or [8] or the human M2DC prepared by the method according to [10] or [11] is administered to a human patient with cancer.

[17] A method for transplantation where an immunological rejection is inhibited, comprising introduction of human expired dendritic cells according to [2] to [5] or human expired dendritic cells obtained by the method according to [9] derived from a human transplantation donor into a human recipient, and then introduction of an organ or a tissue of the human transplantation donor into the human recipient.

[18] The method according to [17] wherein the organ or the tissue is bone marrow.

As sources of the DC, mammals such as human, mouse, cattle, horse, swine, dog and monkey are preferably exemplified, and more preferably the human is exemplified.

In the present invention, the immature DC is converted by any of the following three pathways.

Pathway 1: immature DC→M1DC→expired DC

Pathway 1A (human DC): immature DC→expired DC

Pathway 2: immature DC→M1DC→M2DC (type 1)

Pathway 3: immature DC→M2DC (type 2)

Pathway 1A (human DC): immature DC→expired DC

In Pathways 1 and 2, the M1DC are induced from the immature DC by the natural immune stimulant or a danger signal.

In Pathway 1A, when the human immature DC are stimulated with LPS, they shifts into expired DC which produce IL-10, but clearly activated DC corresponding to M1DC on which the expression of CD80, CD83 or CD86 is rapidly increased and which express IL-12 p40 and IL-10 at a high level are not observed. Therefore, it seems that the human DC shift into the expired DC through either Pathway 1 where the immature DC shift into the expired DC via M1DC (but the expression of surface antigens such as CD80, CD83 and CD86 and the expression of IL-12 p40 and IL-10 are low) or Pathway 1A where the immature DC shift into the expired DC without exhibiting a clear phenotype of the M1DC.

The natural immune stimulant is not particularly limited as long as it induces maturation from the immature DC to the M1DC, and endotoxin (LPS), CpG and the like are exemplified. The natural immune stimulants include those such as LPS, CpG peptide glycan and necrotic cell components, which bind to Toll-like receptor (TLRs) and induce an activation signal. More preferable natural immune stimulants include LPS, CpG and the like.

Induction of M2DC type 1 (mature 2 dendritic cell type 1) from the M1DC in Pathway 2 and the induction of M2DC (type 1) from the immature DC can be performed by the permanent immune potentiator.

In Pathway 1, the shift from the M1DC into the expired DC can be performed without need of a special substance by culturing for about 5 to 100 hours in a usual medium (but including no permanent immune potentiator). Even when the culture is performed in the presence of the natural immune stimulant, the M1DC shift into the expired DC by similarly culturing for about 5 to 100 hours.

The immature DC may be prepared by inducing from bone marrow cells or stem cells capable of shifting into the immature DC using an appropriate inducer, or the immature DC can be directly obtained from spleen. For example, it is possible to induce the immature DC by treating the bone marrow cells with GM-CSF. It is also possible to isolate and use cells such as monocytes capable of shifting into the immature DC from blood. The immature DC, expired DC, M1DC or M2DC (types 1 and 2) can be isolated by a cell sorter after fluorescence labeling or staining. Specifically, in mouse DC, the expired DC and the M2DC can be separated by the cell sorter after staining with CD86. In human DC, the expired DC and the M2DC can be separated by the cell sorter after staining with CD80 or CD83.

Likewise, the immature DC and the M1DC can be separated by the cell sorter after staining with CD80, CD83 or CD86.

Furthermore, the expired DC from the mouse can be separated and purified by sorting CD86-low cells after stimulated with LPS for 48 hours using the cell sorter. The expired DC from the human can be separated and purified by sorting CD80-low cells or CD80-low and CD86-high cells after stimulated with LPS for 48 hours using the cell sorter.

The permanent immune potentiator is not particularly limited as long as it is an activator which can induce the permanently activated mature DC (M2DC) from the immature DC or the M1DC. For example, anti-CD40 antibody (including polyclonal and monoclonal antibodies) which binds to CD40 on the DC to activate the DC, helper T cells which express CD40 ligand (CD154), anti-IL-10 antibody and anti-IL-10 receptor antibody (including polyclonal and monoclonal antibodies) which block the action of IL-10, picibanil (OK432), TNFα, and the like are exemplified. Preferably, the anti-CD40 monoclonal antibody and the anti-IL-10 monoclonal antibody are exemplified. For the human DC, picibanil (OK432) and the anti-CD40 monoclonal antibody can be preferably used.

The M1DC shift into the expired DC by IL-10 produced by themselves, but shift into the M2DC in the presence of the anti-IL-10 antibody or the other permanent immune potentiator (e.g., anti-CD40 mAb).

For the first time, the present invention provides the expired DC and the M2DC.

Characterization of Expired DC

The expired DC have one or more natures as shown below.

(E1) The Expired DC do not Shift into the Mature Type by the Action of the Natural Immune Stimulant and the Permanent Immune Potentiator.

The immature DC are activated with the natural immune stimulant to once shift into the M1DC, and without being affected by the action of the permanent immune potentiator, they shift into the expired DC. Once shifting into the expired DC, even when treated with the natural immune stimulant and the permanent immune potentiator, the expired DC do not change.

(E2) The Expired DC Have the Same Shape as that of the Immature DC.

The expired DC have no spine-shaped dendrites, and have the morphologically same appearance as that of the immature DC.

(E3) The Expired DC Express IL-10.

The expired DC express and secret IL-10. The immature DC do not express IL-10 to an meaningful extent, and the M1DC express IL-10 at a high level, but the expression level of IL-10 by the expired DC shifted from the M1DC is obviously reduced than that by the M1DC, and the expired DC express IL-10 at ½ or less, e.g., about ⅓ to 1/100 quantitatively lower than the M1DC.

Likewise, the expired DC obtained by treating the human immature DC with LPS express IL-10 weakly.

(E4 and E5) The Expired DC Express CD80, CD83 and CD86 at a Low Level (CD80^{low}/CD83^{low}/CD86^{low})

In the DC derived from the mouse, CD86 is expressed at an obviously higher level on activated DC (M1DC and M2DC) than on the immature DC, and the expired DC express CD86 at a level as low as that on the immature DC (CD86^{low}).

Meanwhile in the human DC, through a mild change in quantity of CD80 and/or CD83 expression, the immature DC are changed into the expired DC with low expression (CD80^{low}/CD83^{low}).

(E6) The Expired DC has a Phagocytic Activity for Microbeads at the Same Level as that of the Immature DC.

A phagocytic activity for the microbeads is very low in the activated DC (M1DC and M2DC), but the expired DC have the high phagocytic activity for the microbeads.

(E7) The Expired DC Express MHC class II at a Low Level.

The expired DC express MHC class II at the level as low as that on the immature DC, and this is distinct from the activated DC (M1DC and M2DC) which express MHC class II at a high level. (E8) The Expired DC do not Activate Unreacted T Cells in the Presence of an Antigenic Peptide.

The M2DC induce (activate) the unreacted T cells (CTL) in the presence of the antigenic peptide, but the expired DC do not induce (activate) the unreacted T cells (CTL) in the presence of the antigenic peptide, and rather induce T cell anergy.

(E9) The Expired DC Express TLR4/MD2 at a Lower Level than the Immature DC.

Since the expression level of TLR4/MD2 is low, it is predicted that LPS does not act any more.

Characterization of M2DC

The M2DC have one or more natures as shown below.

(M2-1) The M2DC Have Projecting Dendrites and Form Aggregation Clusters.

As shown in FIG. 2a, the M2DC have projecting dendrites and form aggregation clusters. The activated DC (M1DC and M2DC) have dendrites, and the M2DC have more dendrites than the M1DC.

(M2-2) The M2DC are Capable of Activating Unreacted Cytotoxic T Cells (CTL).

For example, it has been demonstrated that CTL specific for OVA peptide are activated by the M2DC because when the mouse is immunized with the M2DC which have incorporated OVA protein, cells with OVA antigen among target cells transferred into the mouse are specifically eliminated.

(M2-3) The M2DC have Stable Properties Under the Action of Anti-CD40 Monoclonal Antibody.

When cultured in the presence of anti-CD40 monoclonal antibody, the immature DC and the M1DC are changed into the M2DC, but the M2DC are not changed.

(M2-4) The M2DC Show a High Expression Level of at Least One Member Selected from the Group Consisting of CD80, CD83 and CD86.

For example, on the human M2DC obtained by treating the human immature DC with OK432 and anti-CD40 monoclonal antibody, expression levels of CD80 and CD83 are remarkably increased (about 100 times in MF1 in FIG. 10). Therefore, CD80 and CD83 are important indicators to specify the human M2DC. Meanwhile on the mouse M2DC, CD86 is the important indicator, the expression level of CD86 is high on the mouse M2DC and low on the expired DC.

(M2-5) The M2DC Express FcγR at a Low Level (FcγR^{low}).

As shown in FIG. 2b, the M1DC treated with LPS for 24 hours express FcγR at a high level whereas the M2DC treated with anti-CD40 mAb for 24 hours express FcγR at a low level (FcγR^{low}).

(M2-6) The M2DC Express MHC-I at a High Level (MHC-I^{high}).

The expression level of MHC-I is low on the immature DC, and high on the M2DC at a distinct level.

(M2-7) The M2DC express MHC-II at a high level (MHC-II^{high}).

The expression level of MHC-II is high on the M2DC (MHC-II^{high}), and low on the immature DC.

(M2-8) The M2DC Secret (Express) IL-12 p40 at a high level.

Similarly to the M1DC, the M2DC express IL-12 p40 at a high level, and have a potent immunostimulation ability.

Herein, the expired CD “having the same shape as that of the immature CD” means specifically that the expired DC scarcely or does not form aggregation at all as shown in FIG. 2a, is an adherent cell having a spherical, elliptical or rhomboid shape, and there are few formations of spine dendrites and clusters. It appears that the extent of spine dendrite and cluster formation is in proportion to the expression level of CD86 (mouse) or CD80/CD83 (human).

For mammalian cells other than the mouse and the human, the expression level of at least one member of CD86, CD80 and CD83 is changed.

The “low expression levels of CD86/CD83/CD80 (CD86^{low}/CD83^{low}/CD80^{low})” mean that the cell has an absorbance or a fluorescent intensity at 10 times or less (usually 1 to 10 times, particularly 3 to 8 times) when using anti-CD86 antibody/anti-CD83 antibody/anti-CD80 antibody conjugating a marker such as a pigment substance (e.g., phycoerythrin) or a fluorescent substance (e.g., FITC), the expired DC are reacted therewith, and compared to a control (using only the expired DC and not using anti-CD86 antibody/anti-CD83 antibody/anti-CD80 antibody conjugating the marker). The M1DC and the M2DC which “express CD86/CD83/CD80 at high levels (CD86^{high}/CD83^{high}/CD80^{high}) have the absorbance or the fluorescent intensity at about 15 to 100 times and particularly about 50 to 100 times the expired DC (about 50 to 800 times and particularly about 200 to 500 times the control). CD86^{high}/CD83^{high}/CD80^{high} indicates those which exhibit the absorbance or the fluorescent intensity at about 3 to 100 times, preferably about 10 to 80 times and particularly about 20 to 60 times stronger compared to CD86^{low}/CD83^{low}/CD80^{low}.

The expired DC of the present invention has the same shape as that of the immature DC, and is similar to the immature DC in terms of low expression levels of CD86/CD83/CD80 and having the phagocytosis against the microbeads, but is different from the immature DC in the following points.
(i) The expired DC does not shift into the mature DC (M1DC or M2DC) even when treated with the natural immune stimulant such as LPS and the permanent immune potentiator such as anti-CD40 mAb.
(ii) The expired DC expresses IL-10, IL-6 and TNFα at higher levels than the immature DC, and releases them. In particular, the production of immunosuppressive IL-10 seems to be important.
(iii) The expired DC expresses MHC class I (MHC class I/peptide complex) at a high level.
(iv) The expired DC does not activate an unreacted T cell.
(v) The level of TLR4/MD2 is lower in the expired DC than in the immature DC.

Differences of surface antigens on the immature DC, expired DC, M1DC and M2DC are shown in the following Table.

| | Immature | | M2DC | Expired |
|---|---|---|---|---|
| | DC | M1DC | (type1, type2) | DC |
| MHC-I | Low | High | High | High |
| MHC-II | Low | High | High | Low |
| CD80/CD83(human) | Low | High | High | Low |
| CD86(mouse) | | | | |
| FcγR | High | High | Low | High |

The expired DC are elliptical adherent cells with less clusters of the dendrites, which form no aggregation, and exhibit a similar appearance to the immature DC, but these can be clearly distinguished from the M1DC and M2DC which form aggregation clusters and manifest projecting dendrites. The number of the projecting dendrites is more on the M2DC than on the M1DC.

Since the immature DC express CD80/CD83/CD86 at low levels, when introduced into a recipient, it is likely to induce anergy of killer T cells and helper T cells, but it is more likely that the immature CD are activated in the recipient to shift the mature DC (M1DC and M2DC) and stimulate the immune system.

Meanwhile, the expired DC does not return to the mature DC again, and is stable. Therefore, when the expired DC of the donor have been previously administered to the recipient, the administration induces anergy in both killer T cells and helper T cells in the recipient, which are capable of rejecting a graft of the donor. Thus when the organ or the tissue of the donor is then transplanted, it becomes possible to inhibit the immunological rejection. For example, in the case of organ transplantation or bone marrow transplantation, generally it is required that five to six in 6 type HLA are matched. However, it becomes possible to transplant the organ or the bone marrow from the donor with low compatibility of the HLA to the recipient by previously administering the expired DC of the donor to the recipient, and thus it becomes easy to perform the organ transplantation and the bone marrow transplantation.

It is possible to confirm by an MLC (mixed lymphocyte reaction culture) method that the expired DC of the present invention inhibit the immunological rejection. Specifically, it can be confirmed that the expired DC derived from the donor inhibit the immunological rejection by the following procedure. When parts of spleen tissues (before the administration of the expired DC) of the donor and the recipient are taken out and spleen cells in the tissues are mixed and cultured, the number of T cells is increased. However, when the expired DC derived from the donor are administered to induce the anergy of the killer T cells and optionally the helper T cells in the recipient and then the MLC is performed, the number of the T cells is not increased. Although data are not shown, the present inventors have confirmed that the MLC reaction was not caused when the bone marrow from the donor had been transplanted to the recipient after administering the expired DC from the donor to the recipient.

Organs such as heart, liver, kidney, lung, small intestine and pancreas, and further bone marrow are exemplified as the transplanted organs and tissues.

Furthermore, the expired DC is useful not only for the inhibition of graft rejection but also as a therapeutic agent of allergy and autoimmune diseases.

It is possible to prepare the M2DC from the immature DC via the pathway 2 or the pathway 3. The M2DC (type 1) prepared via the pathway 2 express IL-12 (particularly IL-12 p40) at a significantly higher level than the M2DC (type 2) prepared via the pathway 3. They are different in this point. Therefore, it seems that the M2DC (type 1) has a potent immunopotentiation effect.

Differently from the unstable M1DC, the M2DC is stable and does not shift to the expired DC. Therefore, for example, the M2DC (type 1 is more effective) are induced via the pathway 2 or 3 from the immature DC (including those induced from bone marrow cells and blood cells) obtained from a patient with cancer, and these M2DC are returned into the patient with cancer to activate the killer T cells and the helper T cells in the patient with cancer, thereby it becomes possible to prevent cancer metastasis and treat the cancer. Furthermore, the M2DC is effective as an anti-pathogen agent for various pathogens (hepatitis A, B, and C viruses, AIDS virus, influenza virus, and the like). The types of the pathogen are not limited as long as a peptide derived therefrom is available.

The cancers subjected to the treatment are not particularly limited, and include, for example, head and neck cancer, esophageal cancer, stomach cancer, colon cancer, rectal cancer, hepatic cancer, gall bladder/bile duct cancer, pancreatic cancer, lung cancer, breast cancer, ovarian cancer, urinary bladder cancer, prostatic cancer, testicular tumor, osteosarcoma/soft tissue sarcoma, malignant lymphoma, leukemia, uterine cervical cancer, cutaneous cancer, brain tumor and the like.

The present inventors herein described rapid maturation and subsequent expiration of the immature DC after the stimulation with the natural immune stimulant. The expired DC is similar to the immature DC except for the expression of MHC class I at a high level, production of IL-10 at a large amount, and not being further activated. Such expired DC could not stimulate the unreacted CTL, and rather induced the anergy in the CTL clone. The stimulation with the permanent immune potentiator through CD40 and IL-10 inhibited the LPS-induced shift into the expired phenotype, and consequently brought the acquisition of the mature phenotype (M2DC) different from the phenotype (M1DC) observed in the early stage after the stimulation with the natural immune stimulant. These phenomena were also observed in vivo.

These results provide new procedures for immune regulation.

### BRIEF DESCRIPTION OF THE DRAWINGS

FIG. 1 shows time course of CD86 expression on bone marrow DC during maturation.
[a] Immature DC derived from bone marrow of C57BL/6 mice were cultured with anti-CD40 mAb, LPS (lipopolysaccharide) or TNFα, and were analyzed after 8 and 24 hours using a flow cytometry. The cells were stained with anti-CD11c-FITC, anti-CD86-PE (phycoerythrin) and propium iodide (PI). These events were gated within CD11c⁺ and PI^{low}.
[b] Six hours after the stimulation with LPS, CD11c⁺DC were isolated by magnetic selection, and cultured in the presence or absence of LPS or anti-CD40 mAb for 24 hours. The cells were stained with anti-CD86-PE and PI, and gated within PI^{low} population. A viability was 85.3% (medium control), 85.8% (LPS) or 89.7% (anti-CD40).
[c] Expression patterns of CD86 by two different DC populations after the secondary stimulation. The immature DC were stimulated using LPS or anti-CD40 mAb for 48 hours, and then were restimulated using LPS or anti-CD40 mAb for additional 24 hours. These data are representatives of three or more independent experiments having similar results.

In FIG. 1a, control (immature DC), anti-CD40 (8 hrs: mixture of immature DC and M2DC type 2), anti-CD40 (24 hrs: M2DC type 2), LPS (8 hrs: M1DC), LPS (24 hrs: expired DC), TNFα (8 hrs: mixture of immature DC [major] and M2DC type 2 [minor]), and TNFα (24 hrs: mixture of immature DC and M2DC type 2).

In FIG. 1b, expired DC were obtained in CD11c⁺DC (no anti-CD40 mAb and LPS, 24 hrs) and in CD11c⁺DC+LPS (24 hrs), and M2DC type 1 were obtained in CD11c⁺DC+anti-CD40 mAb (24 hrs).

In FIG. 1c, left columns indicate that the expired DC 48 hours after the stimulation with LPS remain the expired DC even when exposed to anti-CD40 and LPS, and right columns indicate that the M2DC (type 1) 48 hours after the stimulation with anti-CD40 remain the M2DC even when exposed to anti-CD40 and LPS.

FIG. 2 shows phenotypic distributions of bone marrow during maturation.
[a] Cellular morphology of 4 different DC subtypes: immature DC (unstimulated), M1DC (cultured with LPS for 6 hours), expired DC (cultured with LPS for 24 hours) and M2DC (cultured with anti-CD40 mAb for 24 hours).
[b] Expression patterns of CD86, MHC class I (H-2K^{k}), MHC class II (H-2A^{k}) and FcγRII/III on immature DC derived from (B6×C3H) F1 mice at 6 or 24 hours after the stimulation with LPS or anti-CD40
[c] Difference of expression levels of TLR4/MD2 on immature DC (white profile) or expired DC (gray profile).
[d] Phagocytic capacity of immature DC, expired DC and M2DC. The cells were cultured with FITC-labeled beads for 8 hours. These data are representatives of three independent experiments having similar results.

In FIG. 2b, LPS 6h (M1DC), LPS 24h (expired DC), anti-CD40 (6 h: mixture of immature DC and M2DC type 1) and anti-CD40 (24 hrs: M2DC type 1).

FIG. 3 shows cytokine production profiles of DC subsets. Immature DC, expired DC and M2DC (type 2), or M2DC (type 1) continuously stimulated with LPS and anti-CD40 mAb were purified by cell sorting.
[a] RNase protection assay of immature DC (1), M1DC (2), M2DC (3) and expired DC (4) (“M” indicates molecular markers).
[b] RT-PCR analysis by real-time quantitative PCR of mRNA levels of IL-6, IL-10 and TNFα in 4 different DC subsets (immature DC [1], M1DC [2], M2DC [type 2; 3] and expired DC [4]) and M2DC (type 1; [5]) continuously stimulated with LPS and anti-CD40 mAb. Copy numbers were standardized for β-actin. These data are representatives of three or more independent experiments having similar results.

FIG. 4 shows functional analyses of DC subsets.
[a] Unreacted CD8⁺ T cells derived from F5 mice were co-cultured with the following DC subsets to which NP₃₆₆₋₃₇₄ at several different concentrations had been applied for 48 hours: immature DC (black profile), expired DC (gray profile) and M2DC (white profile). These data are representatives of four independent experiments having similar results.
[b] A T cell clone (4G3) specific for OVA₂₅₇₋₂₆₄/K^{b} was co-cultured with expired DC and OVA₂₅₇₋₂₆₄ (10 μg) for 48 hours, and then further cultured with MMC-treated B6 spleen cells to which the peptide had been applied (titrated concentrations) for further 24 hours, and the response was measured. These data are representatives of two independent experiments having similar results.

FIG. 5 shows differentiation and maturation in vivo of DC.
[a] Immature DC stimulated with LPS were labeled with CFDA-SE, and injected into DO.11.10 mice (with or without OVA₃₂₃₋₃₃₉). After 48 hours, DC in lymph nodes (LNs) and spleen (SPL) were stained with anti-CD86 antibody. CFSE positive cells were analyzed. These data are representatives of three independent experiments having similar results.
[b] Immature DC were stimulated with peptide glycan III type, CpG ODN or necrotic cell derivatives, LPS or anti-CD40 mAb. The stimulated DC were stained with anti-CD86 6 hours (white profiles) and 48 hours (gray profiles) after the stimulation. These data are representatives of two independent experiments having similar results.
[c] Maturation model of bone marrow DC. The immature DC stimulated with microbial signals through TLR are immediately changed into M1DC which express MHC class II and minor stimulation molecules. The M1DC are bound to Th cells having CD40L, received the signal from CD40 to continuously shift into the M2DC, which maintain the mature phenotype. In the absence of the stimulation from CD40, the M1DC grow to expired DC having down-regulated MHC class II and minor stimulation molecules.

FIG. 6 shows results for effects and action time periods of IL-10 and anti-CD40 antibody.

FIG. 7 shows evaluation (ELISA method) of cytokine secretion depending on induction methods.

FIG. 8 shows evaluation of induction capacity of in vivo killer T cells (CTL).

FIG. 9 shows difference between immature DC and expired DC.

FIG. 10 shows results of stimulating human immature DC derived from monocytes with picibanil (OK432), anti-CD40 antibody or anti-IL-10 antibody. In FIG. 10, a dot line indicates a negative control to which no antibody was added.

### BEST MODE FOR CARRYING OUT THE INVENTION

The present invention will be described with reference to the following Examples, but the invention is not limited thereto.

### EXAMPLE 1

(1) Detection of Novel DC Subsets

The present inventors observed maturation of DC derived from mice with time, and found that almost of all DC responded to LPS with rapid increase of CD86 expression 8 hours after the stimulation with LPS. On the contrary, the stimulation with anti-CD40 mAb and the stimulation with TNFα induced milder increase of a CD86^{high} population. Interestingly, numerous CD86^{low} cells still remained 24 hours after the stimulation with LPS regardless of early rapid up-regulation of CD86 (FIG. 1a). In order to test whether the CD86^{low} population in the DC stimulated with LPS was derived from a CD11c⁺CD86^{high} population, we purified the CD11c⁺ cells from the DC by magnet-sorting the cells 6 hours after the stimulation with LPS, subsequently washing completely, and incubating the cells using LPS or anti-CD40 mAb for 24 hours. Almost of all CD11c+cells prepared in this way expressed CD86 on their surfaces at a high level (not reach a peak). Most CD11c⁺cells lost the surface expression of CD86 at a high level during secondary culture regardless of the presence or absence of LPS. However, interestingly, many CD11c⁺ cells were still CD₈₆^{high} during the secondary culture in the presence of the anti-CD40 mAb (FIG. 1b). Considering almost no change of the cell number observed during the culture, it is difficult to speculate that death of a large number of the CD₈₆high cells was induced within 24 hours after the stimulation with LPS. Therefore, it is highly likely that LPS induced the rapid up-regulation and subsequent down-regulation of CD86 on the DC within 24 hours.

Then, stability of two types of CD phenotypes (i.e., CD86 up-regulated by the anti-CD40 mAb and CD86 down-regulated by LPS) was examined. Thus, two types of DC subtypes were secondarily stimulated with LPS or anti-CD40 antibody. However, neither phenotypes did not change again (FIG. 1c). These data give the following four conclusions.

(1) LPS induces transiently and unstable maturation of bone marrow DC.

(2) Thereafter, the stimulation with LPS produces a stable population having the CD86^{low} phenotype.

(3) The CD40 stimulation exerts the effect to inhibit the LPS-induced down-regulation of CD86.

(4) The anti-CD40 mAb-induced CD86^{high} DC is the DC with relatively stable phenotype

Based on the above data, the present inventors classified the maturation of murine bone marrow DC into four categories.
(1) Immature DC:

Newly differentiated CD86^{low} phenotype with no stimulation.
(2) Mature DC (M1DC) at the first stage:

Early and transient CD86^{high} phenotype after the LPS stimulation.
(3) Mature DC (M2DC) at the second stage:

Stable CD86^{high} phenotype after anti-CD40 stimulation
(4) Expired DC

Late phase CD86^{low} phenotype after the LPS stimulation.

By morphological evaluation of these four types of CD phenotypes, it was shown that a dendritic shape and a cluster formation were mutually interacted with the CD86 expression. The aggregated M1DC were spiny in appearance whereas in the expired DC, these clusters were decreased with the decrease of dendrites, and they had an appearance which visually saw the immature DC. The M2DC also formed solid clusters having projecting dendrites (FIG. 2a). Forty-eight hours after the LPS stimulation, almost of all CD₈₆^{high} phenotype M1DC acquired the phenotype of the CD86^{low} expired DC, but in that population, a small number of CD₈₆^{high} DC still remained.

(II) Difference of DC Subset Phenotypes

Then, it seemed that the immature DC and the expired DC were very similar both in CD86 expression and morphology, and thus, different points between them were searched. It was demonstrated that the expression level of MHC class I indicated a marker which discriminated both by comparing the expression of surface markers between the DC (immature DC) 6 hours after the stimulating using anti-CD40 mAb and the DC (expired DC) 24 hours after the stimulation using LPS (FIG. 2b). Furthermore, M1DC and M2DC were discriminated by the expression levels of FcγII/III. Additionally, the present inventors made a hypothesis that based on non-reactivity of the expired DC to LPS, LPS receptors (TLR4/MD2) were down-modulated on these cells (Nomura F. et al., J. Immunol., 164:3476-3479, 2000). As expected, the immature DC express TLR4/MD2 at a higher level than the expired DC (FIG. 2c). A phagocytic activity of the expired DC was examined using FITC-labeled microbeads (FIG. 2d). Generally, it has been believed that the activated DC can not capture an antigen (Inaba, K. et al., J. Exp. Med., 178:479-488, 1993). However, although the expired DC had been already activated, they captured the beads as efficiently as the immature DC. In contrast, most cells in the M2DC population could not capture the microbeads.

In order to further identify the different points between four DC subtypes, cytokine RNase protection assay of the sorted cells was performed. LPS-stimulated two subsets (M1DC and expired DC) exhibited very similar cytokine m-RNA patterns. In the expired DC, IL-1 and IL-6 were up-regulated, but signal for IL-12 p40 was weak. In contrast, the M2DC had a quite different pattern (i.e., up-regulation of IL-12 p40 and IL-6, conversely down-regulation of IL-1 and IL-1Ra) (FIG. 3a). In order to compare the cytokine production more precisely, relative copy numbers of mRNA for IL-6 and IL-10, and TNFα in 4 DC subsets and the DC stimulated with LPS then anti-CD40 mAb consecutively were measured by real-time quantitative PCR. After the stimulation with LPS, the expressions of IL-6, IL-10 and TNFα were up-regulated in the M1DC and then slightly down-regulated in the expired DC whereas the up-regulation of IL-6 was scarcely detected and the expression of TNFα and IL-10 at low levels was detected in the M2DC (FIG. 3b). The CD86^{high} mature DC induced from the M1DC by the stimulation using the anti-CD40 mAb exhibited almost the same cytokine profile as that in the M2DC, supporting a concept that the stimulation with anti-CD40 mAb mainly mediates the shift from the M1DC to M2DC. These different cytokine production profiles clearly distinguish four types of the DC subsets.

Since the expression levels of MHC class I were very similar between the M2DC and the expired DC, the present inventors compared abilities to activate CTL of the different DC subsets. Unreacted CD8⁺ T cells (here, this T cell has a D^{b}-specific antigen receptor specific an influenza nuclear protein (NP)₃₆₆₋₃₇₄) derived from an F5 transgenic mouse were cultured with three types of DC subsets (i.e., immature DC, M2DC and expired DC) to which the NP peptides at several types of concentrations had been added. The present inventors thought that the M1DC was not suitable for functional assays because the phenotype of the M1DC was provisional. It was found that the immature DC and the expired DC could not activate the unreacted T cells whereas the M2DC activated the CTL at concentrations of 100 pM or more of the NP₃₆₆₋₃₇₄ (FIG. 4a).

Then, it was examined whether the expired DC could induce the anergy of T cells or not.

A T cell clone (4G3) specific for OVA₂₅₇₋₂₆₄ (peptide derived from ovalbumin [OVA]) was cultured with the expired DC and OVA₂₅₇₋₂₆₄ for 48 hours, and then cultured again with spleen cells of B6 and OVA₂₅₇₋₂₆₄ at various concentrations. These 4G3 cells still responded to Con A culture supernatant, but did not respond any more to OVA₂₅₇₋₂₆₄ at any concentrations examined. This indicates that the expired DC can induce the anergy.

(III) Novel Model of DC Maturation

Based on these data, the present inventors propose the model that the M1DC shifts into the expired DC in the absence of signaling through CD40 whereas the M1DC shifts into the stable mature type (M2DC) when receiving the signal through CD40 by the anti-CD monoclonal antibody (FIG. 5c). The M1DC also differentiates into the stable mature type (M2DC) even when using anti-IL-10 monoclonal antibody in place of the anti-CD40 monoclonal antibody. As a result, it has been found that the CD40 stimulation plays a crucially important role in the selection for the activation in opposition to toleration of the CTL. Meanwhile, it has been reported that heat shock protein 70 of mycobacterium stimulates monocyte-induced DC through CD40. Therefore, it can not ignore that there can be a pathway in which the immature DC directly shifts into the M2DC through the CD40 stimulation (Wang, Y. et al. Immunity 15, 971-983 (2001)). By this model and with reference to the already published reports (Stoll, S. et al. Science 296, 1973-1876 (2002); Ingulli, E. et al. J. Immunol. 169, 2247-2252 (2002); Lee, B. O. et al. J. Exp. Med. 196, 693-704 (2002)), the following scenario in vivo is predicted. Focusing on the inflammation, the immature DC capture an antigen, and receive a signal to cause the differentiation into the M1DC through TLR or proinflammatory lymphokine receptor. The M1DC rapidly migrate into a regional lymph node where they induce the CD40L expression on the helper T cell surfaces. This series of events is accomplished generally within 24 hours, and particularly within 12 hours. Therefore, the activated helper T cells give a signal to the M1DC through CD40, and consequently the differentiation into the M2DC capable of activating the CTL occurs before the final change into the expired DC occurs. When the interaction between CD40 and CD40L is failed, the shift into the expired DC which tolerates the CTL is induced (FIG. 5c). In order to confirm this in vivo, OVA₃₂₃₋₃₃₆ (which activates the helper T cells in DO11.10 mice) was added to CFDA-SE labeled CD11c⁺M1DC induced by LPS, which was then inoculated to DO11.10 mice. After two days, the CD86 expression was analyzed for CFSE-positive cells in the regional lymph nodes and the spleen. Only DC in the lymph node to which OVA₃₂₃₋₃₃₆ had been added expressed CD86 at a high level whereas most DC in the spleen were the DC with immature/expired phenotypes regardless of the presence or absence of the OVA peptide (FIG. 5a). Therefore, it appears that the M1DC interact with the helper T cells particularly in the lymph nodes and subsequently progress to the M2DC.

Finally, the present inventors examined whether the expiration of the M1DC was a general phenomenon or not. The immature DC were cultured with peptide glycan (TLR2 ligand), CpG ODN (TRL9 ligand) and necrotic cells (believed to be an endogenous activation factor and a natural adjuvant derived from an autologous substance: Gallucci, S., Lolkema, M. & Matzinger, P. Nat. Med., 5, 1249-1255 (1999)) for 6 or 48 hours. The anti-CD40 mAb induced the CD86 expression at a high level on the DC after 48 hours but did not induced on the DC after 6 hours (same as shown in FIG. 1). On the contrary, peptide glycan, CpG ODN and the necrotic cells induced the strong expression of CD86 after 6 hours, but CD86 was not obviously expressed on most DC after 48 hours, and the DC stimulated with LPS were similar (FIG. 5b). These data indicate that the maturation model proposed here is a major physiological pathway of the DC maturation.

(IV) Discussion

The new mature DC model of the present invention can solve long-standing problems. First, why is only the anti-CD40 mAb among all reagents which induce DC characterized remarkably by CTL activation ? (Ridge, J. P. et al. Nature 393, 474-478 (1998); Bennett, S. R. et al. Nature 393, 478-480 (1998); Schoenberger, S. P. et al. Nature 393, 480-483 (1998)). The present inventors have thought that the real role of the CD40 stimulation in the CTL induction is maintenance of continuous stability of the mature DC phenotype. In fact, the expression of an MHC class II/peptide complex and the expression of an MHC class I/peptide complex were detected about 8 and 24 hours after the stimulation with LPS (unpublished data). Since the M1DC phenotype exists for several hours in the early stage, the expression level of the MHC class I/peptide complex can not reach the level required for the CTL activation at this stage. The expired DC also have no ability for activating the CTL because CD80/CD86 and IL-12 are scarcely expressed although the MHC class I/peptide complex is retained at a sufficient level. Only the M2DC accomplished 24 hours after the CD40 stimulation or 48 hours after the picibanil+CD40 stimulation could present the MHC class I/peptide complex to the CTL. Taken together, it is likely that major roles of the M1DC and the M2DC are to activate the helper T cells and the CTL (or the helper T cells), respectively.

Second, “can the immature DC alone induce CTL toleration?” The present inventors have identified phenotypically and functionally that the expired DC can be a candidate of tolerance-inducing DC which is functionally the same as the immature DC. It has been believed that the immature DC can exert the effect which causes the tolerance (Hawiger, D. et al. J. Exp. Med. 194, 769-779 (2001); Hugues, S. et al. Immunity 16, 169-181 (2002); Jonuleit, H. et al. J. Exp. Med. 192, 1213-1222 (2001); Liu, K. et al. J. Exp. Med. 196, 1091-1097 (2002)). However, Albert et al. (Albert, M. L. et al. Nat. Immunol., 2, 1010-1017 (2001)) has demonstrated that the DC maturation is required for the induction of cross toleration of CD8⁺ T cells. Furthermore, the CD40 stimulation of the DC dictated the result of not the cross toleration but cross activation. The model of the present invention probably illustrates these studies. Additionally, the expired DC has various characteristics attributed to the ability to decrease the immune responses (including the production of IL-10). Therefore, this subset can be useful for the regulation of antigen specific immune responses.

Finally, it will be illustrated what a mechanism for endotoxin tolerance is. The endotoxin tolerance (Greisman, S. E. et al. J. Exp. Med. 124, 983-1000 (1966)) (induced by continuous exposure to the endotoxin [including LPS]) is probably explained by the rapid change of the phenotypes (from the M1DC to the expired DC) which occurs in the absence of the stimulation through CD40. This concept is partially supported by the expression of TLR4/MD2 at a lower level on the expired DC (FIG. 2b). Furthermore, the studies described in several reports (Wysocka, M. et al. J. Immunol. 166, 7504-7513 (2001); Alves-Rosa, F. et al. Clin. Exp. Immunol. 128, 221-228 (2002)) (the production of IL-12 is inhibited during experimental endotoxin tolerance; and IL-1β is involved in endotoxin tolerance) are also explained by cytokine profiles of the expired DC. Likewise, in large amount necrosis and severe infection (e.g., endotoxin shock), since extremely numerous M1DC exist, the helper T cells can not differentiate all of the M1DC into the M2DC. Consequently, these bring about the increase of the expired DC and phenomena of immunodeficiency corresponding thereto. On the whole, it is concluded that the DC subsets defined here are deeply involved in the regulation of the immune responses.

### EXAMPLE 2

(I) Preparation of Bone Marrow DC

DC were prepared from murine bone marrow as described above (Inaba, K. et al J. Exp. Med. 176, 1693-1702 (1992)). Briefly, cells obtained by removing T cells, B cells and granulocytes using specific antibodies and complement from bone marrow cells of C57BL/6J, BALB/cCr or B6C3F1 mice (SLC) were cultured in a 24-well culture plate in RPMI 1640 (Nacalai Tesque) supplemented with 10% FCS, 4 ng/mL of recombinant murine granulocyte/macrophage colony stimulating factor (rmGM-CSF)(provided by Kirin Brewery Co Ltd.) and 50 μL of β-mercaptoethanol for 6 days. The culture medium was replaced with fresh one every other day. The maturation of the DC was induced by adding 5 μL/mL of anti-CD40 mAb (NM-40-3), 1 μL/mL of LPS (derived from E. coli) (Nacalai Tesque), 1 μL/mL of peptide glycan III type (derived from S. aureus) (Wako), 0.1 μM of phosphothioate-protected CpG ODN (5′-TCCATGACGTTCTTGATGTT-3′; SEQ ID NO:1; Hokkaido System Science), or a derivative of frozen/thawed COS-7 cells at 1×10⁵ (as necrotic cells) to each culture well.

(II) Flow Cytometry

The CD were treated with anti-FcγRII/III mAb (2.4G2), and then stained with fluorescein (FITC)-conjugated anti-CD11c, phycoerythrin (PE)-conjugated anti-CD86 or anti-I-A^{k}, or biotin-conjugated hamster anti-IgG, anti-I-K^{k} (BD PharMingen) or anti-TLR4/MD2 (eBioscience), which were reacted with streptavidin-PE. The stained cells were obtained using FACScan (Becton Dickinson Immunocytometory system).

The results are shown in FIG. 2b.

(III) Phagocytosis Assay

Immature DC, M2DC or expired DC (1×10⁵) sorted using FACSVantage (Becton Dickinson Immunocytometory system) were co-cultured with FITC-labeled 2 μm resin microbeads (5×10⁶) (Sigma). After incubating for 8 hours, the respective DC were analyzed by the FACS machine.

(IV) RNase Protection Assay (RPA)

Total RNA was isolated from each subset of the DC using an RNA separation kit (Roche Diagnostics) in accordance with protocols of the manufacturer. Levels of cytokine mRNA were detected by RPA using RiboQuant kits (BD PharMingen) with mCK-2b probe set. Briefly, the total RNA (2 μg) of each DC subset sorted by the FACSVantage was hybridized with [α-³²P]UTP-labeled antisense ribo probe at 56° C. for 16 hours. After digesting with RNase A and proteinase K, protected RNA fragments were separated on a denaturing sequence gel, and then autoradiography was performed.

(V) RT-PCR

The total RNA (0.5 μg) was used for cDNA synthesis. Reverse transcription (RT) was performed using SuperScript™ II RTase and oligo-(dT)₁₂₋₁₈ primer (Invitrogen) in accordance with the protocols of the manufacturer. In a total volume 20 μL of an RT reaction mixture solution, 1 μL was used as a template for each real-time PCR reaction. Primers and a hybridization probe were designed and synthesized using Primer 3 software (http://www-genome.wi.mit.edu/cgi-bin/primer/primer3_www.cgi) (Hokkaido System Science). This probe was modified to bind a reporter dye at the 5′ terminus or bind a quenching agent at the 3′ terminus. Sequences of the oligonucleotides were mβ actin (forward direction: 5′-ggccaggtcatcactattgg-3′ [SEQ ID NO:2]; reverse direction: 5′-atgccacaggattccatacc-3′ [SEQ ID NO:3]), a probe (5′Fam-tcagggcatcggaaccgctc-Tamra3′ [SEQ ID NO:4]), mIL-6 (forward direction: 5′-cttcacaagtcggaggcttaa-3′ [SEQ ID NO:5]; reverse direction: 5′-cagaattgccattgcacaac-3′ [SEQ ID NO:6]), a probe (5′Fam-tcatttccacgatttcccagagaaca-Tamra3′ [SEQ ID NO:7], mIL-10 (forward direction: 5′-cctgggtgagaagctgaaga-3′ [SEQ ID NO:8]; reverse direction: 5′-gctccactgccttgctctta-3′ [SEQ ID NO:9]), a probe (5′Fam-aatcgatgacagcgcctcagcc-Tamra3′ [SEQ ID NO:10], and TNFα (forward direction: 5′-ccagaccctcacactcagatc-3′ [SEQ ID NO:11]; reverse direction: 5′-cacttggtggtttgctacga-3′ [SEQ ID NO:12]), a probe (5′Fam-aattcgagtgacaagcctgtagcccac-Tamra3′ [SEQ ID NO:13]). Polymerase chain reactions (PCR) were performed using LightCycler (registered trademark) (Roche Diagnostics) as described previously (Stordeur, P. et al. J. Immunol. Method. 259 55-64 (2002)). Briefly, a reaction mixture solution of a final volume 20 μL was made using 2 μL of FastStart DNA Master Hybridysation Probes (Roche Diagnostics), 1 μL of the hybridization probe (4 pmol/μL) and the primers in forward and reverse directions at an appropriate concentration. After an initial denature step (95° C. for 10 min), a temperature cycle (95° C. for 0 second, 60° C. for 20 seconds) was started.

Total 45 cycles were performed. At the end of each cycle, fluorescein was read out using F1/F2. All amplifications were performed three times. Quantification was performed by calculated values from a standard curve. All results were standardized for β-actin.

(VI) T Cell Proliferation Assay

Unreacted T cells derived from a TCR transgenic mouse (F5) which had RAG-1 deficient (Corbela, P. et al. Immunity 1, 269-276 (1994)), was restricted to D^{b} and was specific for NP₃₆₆₋₃₇₄ were purified using a nylon wool column. The T cells (2×10⁵/well) were cultured with each DC subset (5×10⁵/well) for 3 days. The response was determined by uptake of ³H-thymidine. In order to observe anergy-inducing ability of the expired DC, a K^{d}-restricted T cell clone specific for OVA₂₅₇₋₂₆₄ (1×10⁶/well)(4G3; Sykulev, Y. et al. Proc Natl. Acad. Sci. U S A 91, 11487-11491 (1994)) was cultured with the expired DC (1×10⁵/well) in the presence of 10 μL of OVA₂₅₇₋₂₆₄ in a 24-well plate for 2 days. Then, 4 G3 cells (1×10⁵/well) were washed, and cultured with spleen cells (as antigen presenting cells) treated with mitomycin C in the presence of OVA₂₅₇₋₂₆₄ at titrated concentrations. The response was determined by the uptake of ³H-thymidine.

(VII) DC Maturation Assay in Vivo

CFDA-SE (Molecular probes)-labeled and LPS-stimulated DC (1×10⁷) (OVA₂₅₇₋₂₆₄ peptide [Hokkaido System Science] had been applied or had not been applied) were subcutaneously injected in four footpads of OD11.10 mice (Murphy, K. A. et al. Science 250, 1720-1723 (1990)). Two days after the injection, lymph nodes and spleen were digested at 37° C. for 30 min using collagenase with low resolvability. Then, CFSE-positive cells were stained with anti-CD86 antibody, and analyzed as the above.

### EXAMPLE 3

1. The Change of Phenotypes in Dendritic Cells Highly Depended on IL-10, which was Different in Action Phase from Stimulant Anti-CD40 Antibody Having an Antagonistic Action Thereto.

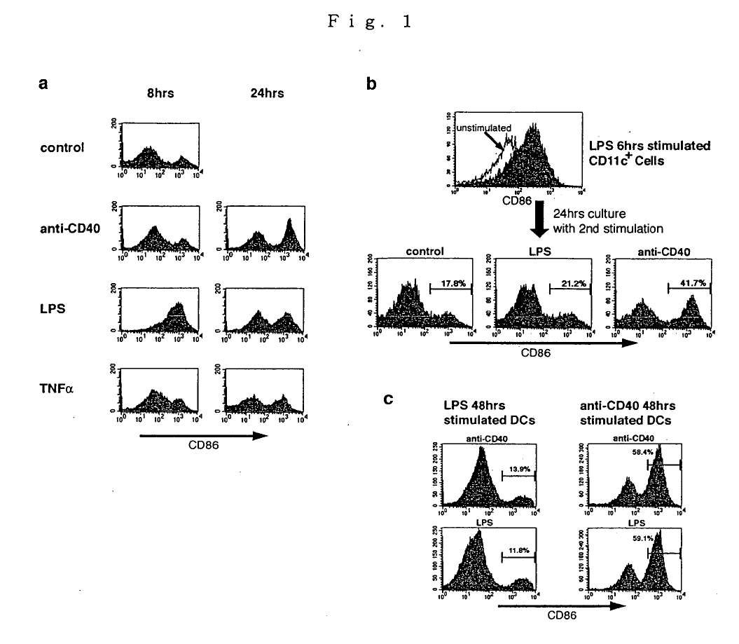

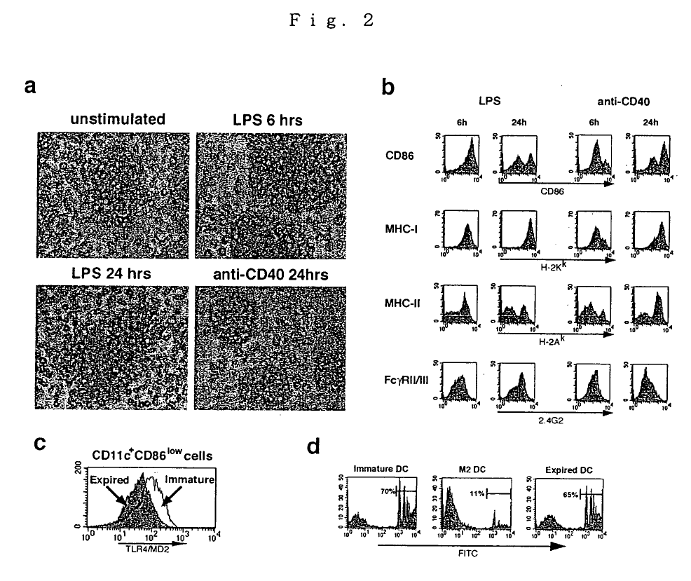

Murine immature dendritic cells were stimulated with LPS, and the expression change of a substimulant molecule CD86 with time was analyzed. The expression of the CD86 molecules on the dendritic cells was transiently increased by the stimulation with LPS, but thereafter gradually decreased (FIG. 6, upper panels). At that time, when anti-IL-10 receptor antibody which blocked the action of IL-10 was added at the start of the reaction, numerous mature dendritic cells remained even after 30 hours (FIG. 6, middle panels). In order to confirm whether this effect of IL-10 acts before the transient activation or occurs after the transient activation, the medium was changed 8 hours after the start of the LPS stimulation, and then the anti-IL-10 receptor antibody (aIL-10) was added. As a result, blockage of IL-10 action after the transient activation did not result in prevention of the expiration (FIG. 6, lower panels). Therefore, it has been found that IL-10 acts in the early stage from the dendritic cells being stimulated to being transiently activated. It has been also found that anti-CD40 antibody affects even when added after the transient activation (FIG. 1, lower panel). These results indicate that it is possible to control whether the dendritic cells are induced to the phenotype of immune activation or the phenotype of immune suppression by temporally or quantitatively regulating the stimulation through IL-10 and CD40.

2. Reevaluation of Cytokine Production by Functional Induction Methods of Dendritic Cells

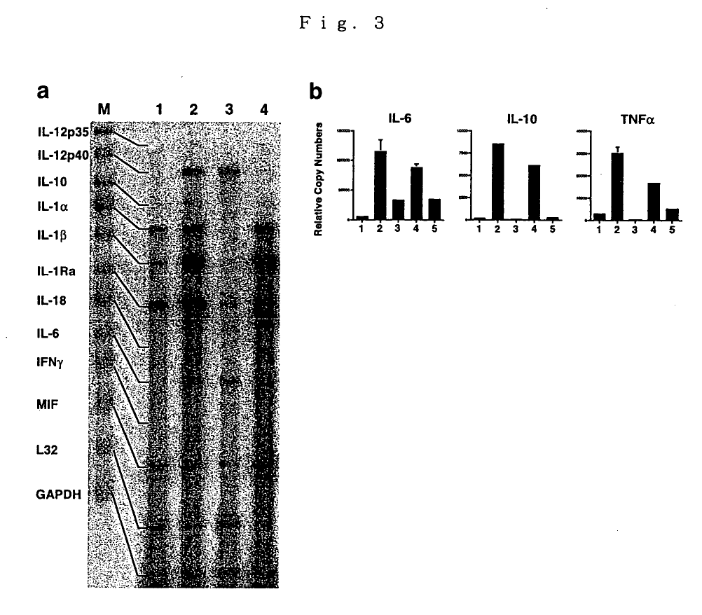

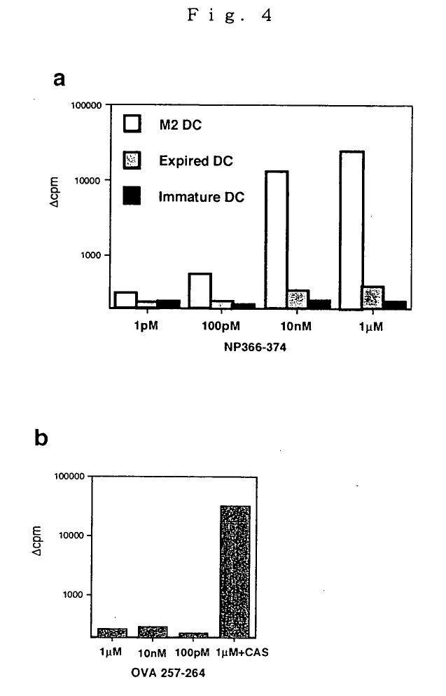

Difference of cytokine production was shown by adding anti-CD40 antibody (aCD40) or anti-IL-10 antibody or both thereof in addition to LPS or CpG when the dendritic cells were functionally induced to an activated type or an expired type. As the activated type, those expressing CD86 at a high level were isolated by a flow cytometer, and cultured. As the expired type, those expressing CD86 at a low level were isolated and cultured similarly. Consequently, for the activated type dendritic cells (M2DC), the production of IL-12 p40 was high and the production of IL-10 which was a suppressive cytokine was low in the group to which both anti-CD40 antibody (aCD40) and anti-IL-10 antibody (aIL-10) had been added. Thus, the combination of CpG+anti-CD40 antibody+anti-IL-10 antibody was the most effective for the production of IL-12. For the difference between the transiently activated type (M1DC) and the activated type (M2DC), the M1DC produced both IL-10 and IL-12 whereas the M2DC produced only IL-12 and IL-10 produced by the M2DC was below a significantly measurable value. Meanwhile, in the case of the expired DC, no secretion of IL-12 was observed and IL-10 was produced at a small amount (FIG. 7).

3. Functions in Vivo of Activated Type (M2DC) or Expired Type Dendritic cells

Experimental protocol: Activated type or suppressive type dendritic cells in which egg albumin (OVA) protein antigen was incorporated are subcutaneously injected into four footpads of C57BL/6 (B6) mice. The dendritic cells incorporating the OVA protein antigen and stimulated with LPS for 6 hours is further administered in the four footpads after one week. After additional one week, spleen cells from different B6 mice are labeled with different fluorescent intensities. The cells with high fluorescent intensity are pulsed with 10 μM of OVA protein peptide (amino acid sequence: SIINFEKL), and the cells with low fluorescent intensity are pulsed with NP peptide (amino acid sequence: ASNENMDAM) which is not related to the OVA protein. They are mixed at 1:1, and 1×10⁶ thereof are intravenously administered to all mice immunized with the dendritic cells. After 10 hours, spleen cells of the immunized mice are removed, and the cells labeled with fluorescence are analyzed by the flow cytometer.

Changed Points of Dendritic Cell Induction Protocol

Based on the results of 1, as the activated type dendritic cells, those obtained by culturing the immature dendritic cells in the medium containing LPS (1 μg/mL), anti-CD40 antibody (10 μg/mL) and anti-IL-10 antibody (10 μg/mL) for 30 hours, and purifying a fraction in which CD86 was expressed at a high level by the flow cytometer were used. As the suppressive type dendritic cells, those obtained by culturing in the medium containing LPS (1 μg/mL) or CpG (0.1 μM) for 30 hours, and purifying a fraction in which CD86 was expressed at a low level by the flow cytometer were used.

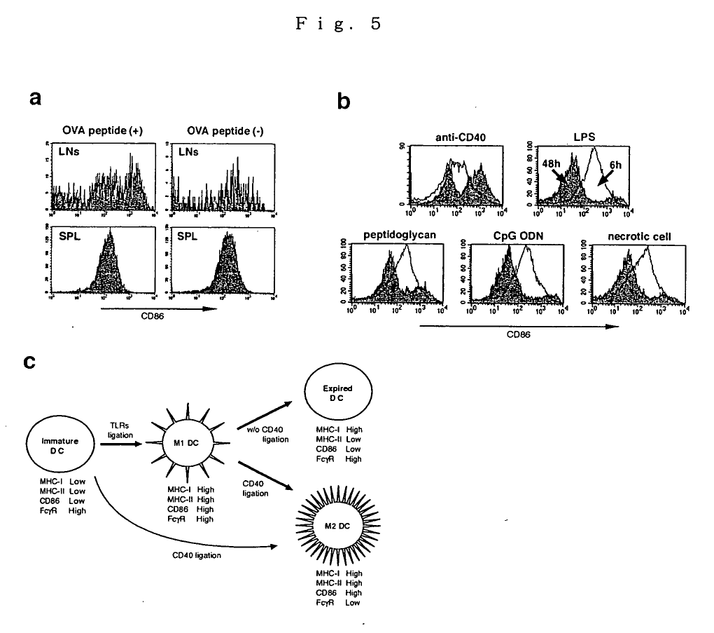

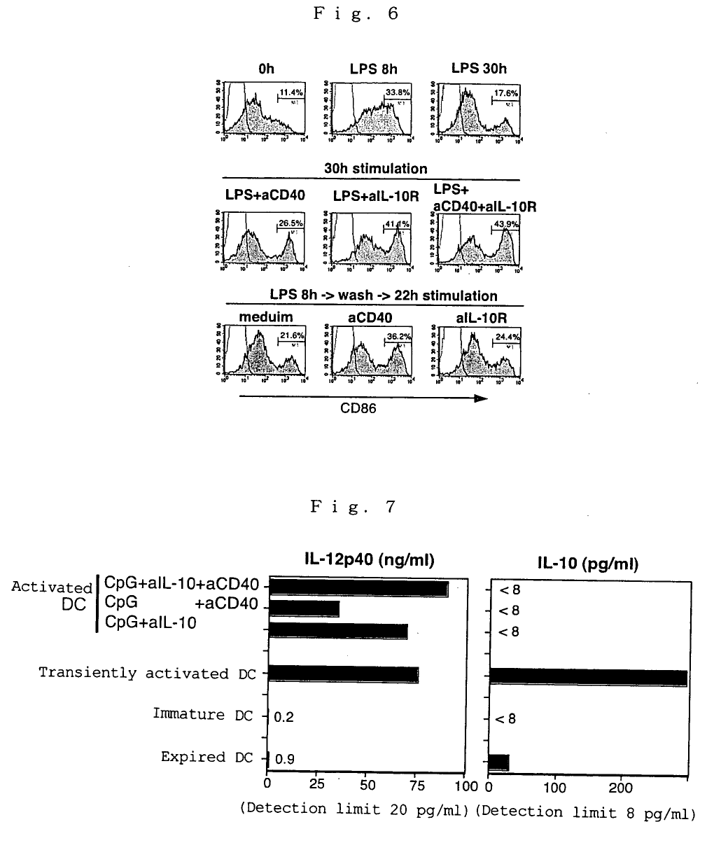

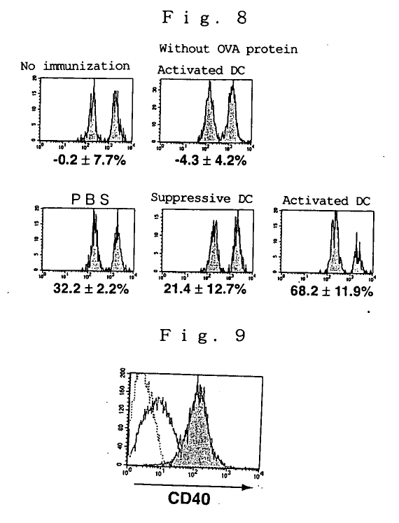

Results: Since the dendritic cells have incorporated the OVA protein, the mouse has been immunized with the OVA antigen. If immunity has been induced, a cell population having the high fluorescent intensity is eliminated. First, it was confirmed by dendritic cells from the untreated mouse and the activated type dendritic cells not primed with OVA whether the experimental system worked well or not. Consequently, no elimination was observed for both cells (FIG. 8 upper panels). In PBS group where only one immunization had been given, about 30% antigen-specific elimination was observed (FIG. 8, lower left panel), but in the group where the immunization with the activated type dendritic cells had been previously given, about 70% elimination was observed, indicating that the effect was facilitated (FIG. 8, lower right panel). Meanwhile, in the group where the immunization with the suppressive type dendritic cells had been previously given, the antigen specific elimination was inhibited (FIG. 8, lower middle panel). From the above results, the effects of the active type and suppressive type dendritic cells on application in vivo have been demonstrated.

4. For Difference Between Immature Dendritic Cells and Expired Dendritic Cells

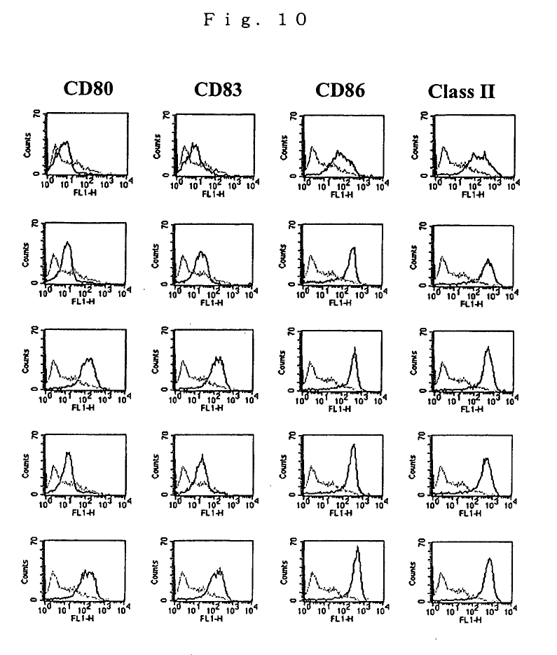

Bone marrow dendritic cells were purified with CD11c magnetic beads, and the expression of CD40 on those (immature dendritic cells) before the stimulation and those (expired dendritic cells) obtained by stimulating with LPS for 48 hours was evaluated by staining with HM40-3 antibody. As a result of analysis by the flow cytometer (FIG. 10), although the immature dendritic cells expressed CD40 molecules, the expired dendritic cells expressed CD40 molecules 10 to 20 times in fluorescent intensity.

### EXAMPLE 4

Induction of Human Activated Dendritic Cells

1. Induction Method

Monocytes are isolated from peripheral blood of a patient with cancer by a density gradient centrifuge method.
The monocytes at 2×10⁷ are suspended in 10 mL of a medium containing 5% human AB serum, and subsequently, left stand in a 100 mm plastic plate for 2 hours.
Suspended cells are removed, the medium containing 5% human AB serum is added thereto, and cells which adhere to the plastic plate are cultured in the presence of 5 ng/mL GM-CSF and 100 ng/mL IL-4 for 7 days.
The medium is removed, a new medium containing OK432 (0.1 KE/mL), anti-CD40 antibody (2.5 μg/mL) or anti-IL-10 antibody (10 μg/mL) is added and the culture is performed for 48 hours.
After the completion of the culture, surface markers on induced dendritic cells are examined by the flow cytometry.

In FIG. 10, from the upper, dendritic cells with no addition (immature DC), dendritic cells induced by the addition of OK432 alone (expired DC), OK432/anti-CD40 antibody (M2DC type 1), OK432/anti-IL-10 antibody (M2DC type 1) and OK432/anti-CD40 antibody/anti-IL-10 antibody (M2DC type 1).

2. Results

Compared to the induced cells with no addition (immature DC), in human activated DC, all expression levels of CD80, CD83, CD86 and MHC class II were increased by the addition of OK432. By further addition of anti-CD40 antibody, the expression levels of CD80 and CD83 were remarkably increased (about 100 times in MFI). These increases in expression were not observed by the addition of anti-IL-10 antibody.

The stimulation with OK432+anti-CD40 antibody enhanced the expression level of CD80 most strongly, and this seems to be important in terms of clinical application.

### REFERENCES

1. Nomura, F. et al. J. Immunol. 164, 3476-3479 (2000)
2. Inaba, K. et al. J. Exp. Med. 178, 479-488 (1993)
3. Wang, Y. et al. Immunity 15, 971-983 (2001)
4. Stoll, S. et al. Science 296, 1973-1876 (2002)
5. Ingulli, E. et al. J. Immunol. 169, 2247-2252 (2002)
6. Lee, B. O. et al. J. Exp. Med. 196, 693-704 (2002)
7. Ridge, J. P. et al. Nature 393, 474-478 (1998)
8. Bennett, S. R. et al. Nature 393, 478-480 (1998)
9. Schoenberger, S. P. et al. Nature 393, 480-483 (1998)
10. Hawiger, D. et al. J. Exp. Med. 194, 769-779 (2001)
11. Hugues, S. et al. Immunity 16, 169-181 (2002)
12. Jonuleit, H. et al. J. Exp. Med. 192, 1213-1222 (2001)
13. Liu, K. et al. J. Exp. Med. 196, 1091-1097 (2002)
14. Albert, M. L. et al. Nat. Immunol., 2, 1010-1017 (2001)
15. Greisman, S. E. et al. J. Exp. Med. 124, 983-1000 (1966)
16. Wysocka, M. et al. J. Immunol. 166, 7504-7513 (2001)
17. Alves-Rosa, F. et al. Clin. Exp. Immunol. 128, 221-228 (2002)
18. Inaba, K. et al J. Exp. Med. 176, 1693-1702 (1992)
19. Stordeur, P. et al. J. Immunol. Method. 259 55-64 (2002)
20. Corbela, P. et al. Immunity 1, 269-276 (1994)
21. Sykulev, Y. et al. Proc Natl. Acad. Sci. U S A 91, 11487-11491 (1994)
22. Murphy, K. A. et al. Science 250, 1720-1723 (1990)

1. An expired dendritic cell having characteristics of the following (E1) to (E3):
(E1) not shifting into a mature type due to an action of a natural immune stimulant or a permanent immune potentiator;
(E2) having the same shape as immature DC; and
(E3) expressing IL-10.

2. The expired dendritic cell according to claim 1 wherein said dendritic cell is a human dendritic cell.

3. The human expired dendritic cell according to claim 2 having the following characteristics:
(E1′) not shifting into a mature type due to an action of LPS and anti-CD40 monoclonal antibody;
(E2) having the same shape as immature DC; and
(E3) expressing IL-10.

4. The human expired dendritic cell according to claim 3 further having the following characteristics:
(E4) having an expression level of CD80 nearly equivalent to that on the immature DC; and/or
(E5) having an expression level of CD83 nearly equivalent to that on the immature DC.

5. The human expired dendritic cell according to claim 4 further having at least one of the following characteristics:
(E6) having a phagocytic activity for microbeads nearly equivalent to that of the immature dendritic cells;
(E7) expressing MHC class I at a high level;
(E8) not activating unreacted T cells in the presence of an antigenic peptide; and
(E9) expressing TLR4/MD2 at lower level than the immature DC.

6. Permanently activated dendritic cells having the following characteristics:
(M2-1) having projecting dendrites and forming aggregation clusters;
(M2-2) being capable of activating unreacted cytotoxic T cells (CTL);
(M2-3) having stable properties under the action of anti-CD40 monoclonal antibody; and
(M2-4) showing a high expression level of at least one member selected from the group consisting of CD80, CD83 and CD86.

7. The permanently activated dendritic cells according to claim 6 wherein said dendritic cells are cells derived from human, having the following characteristics:
(M2-1) having projecting dendrites and forming aggregation clusters;
(M2-2) being capable of activating unreacted cytotoxic T cells (CTL);
(M2-3) having stable properties under the action of anti-CD40 monoclonal antibody; and
(M2-4′) expressing CD80 and CD83 at high levels.

8. The permanently activated dendritic cell according to claim 7 further having at least one of the following characteristics:
(M2-5) expressing FcγR at a low level (FcγR^{low});
(M2-6) expressing MHC-I at a high level (MHC-I^{high});
(M2-7) expressing MHC-II at a high level (MHC-II^{high}); and
(M2-8) expressing IL-12 p40 at a high level.

9. A method for preparing expired dendritic cells (expired DC) comprising a step of activating immature dendritic cells with a natural immune stimulant to induce transiently activated mature dendritic cells (M1DC), and a step of culturing the M1DC in the absence of a permanent immune potentiator.

10. A method for preparing permanently activated mature dendritic cells (M2DC) comprising a step of treating immature dendritic cells with a permanent immune potentiator.

11. A method for preparing permanently activated mature dendritic cells (M2DC) comprising a step of activating immature dendritic cells with a natural immune stimulant to induce transiently activated mature dendritic cells (M1DC), and a step of culturing the M1DC in the presence of a permanent immune potentiator.

12. A method for preparing transiently activated mature dendritic cells (M1DC) characterized by treating immature dendritic cells with a natural immune stimulant.

13. An anti-cancer agent wherein the human permanently activated dendritic cell (M2DC) according to claim 7 is an active ingredient.

14. An anti-pathogen agent wherein the human permanently activated dendritic cell (M2DC) according to claim 7 is an active ingredient.

15. An immunosuppressive drug wherein the expired dendritic cell according to claims 1 is an active ingredient.

16. A method for treating cancer characterized in that the human permanently activated dendritic cell (M2DC) according to claim 7 is administered to a human patient with cancer.

17. A method for transplantation where an immunological rejection is inhibited, comprising introduction of human expired dendritic cells according to claims 2 derived from a human transplantation donor into a human recipient, and then introduction of an organ or a tissue of the human transplantation donor into the human recipient.

18. The method according to claim 17 wherein said organ or tissue is bone marrow.


SEQUENCE LISTING (13 sequences)

SEQ ID NO: 1 - DNA, 20 bp, mouse
tccatgacgttcttgatgtt

SEQ ID NO: 2 - DNA, 20 bp, mouse
ggccaggtcatcactattgg

SEQ ID NO: 3 - DNA, 20 bp, mouse
atgccacaggattccatacc

SEQ ID NO: 4 - DNA, 20 bp, mouse
tcagggcatcggaaccgctc

SEQ ID NO: 5 - DNA, 21 bp, mouse
cttcacaagtcggaggcttaa

SEQ ID NO: 6 - DNA, 20 bp, mouse
cagaattgccattgcacaac

SEQ ID NO: 7 - DNA, 26 bp, mouse
tcatttccacgatttcccagagaaca

SEQ ID NO: 8 - DNA, 20 bp, mouse
cctgggtgagaagctgaaga

SEQ ID NO: 9 - DNA, 20 bp, mouse
gctccactgccttgctctta

SEQ ID NO: 10 - DNA, 22 bp, mouse
aatcgatgacagcgcctcagcc

SEQ ID NO: 11 - DNA, 21 bp, mouse
ccagaccctcacactcagatc

SEQ ID NO: 12 - DNA, 20 bp, mouse
cacttggtggtttgctacga

SEQ ID NO: 13 - DNA, 27 bp, mouse
aattcgagtgacaagcctgtagcccac